(19) United States
(12) Patent Application Publication
(10) Pub. No.: US 2023/0413877 A1
(43) Pub. Date: Dec. 28, 2023

(54) Dissolving Oral Implement Assembly

(71) Applicant: Felicia Evans, Lancaster, TX (US)

(72) Inventor: Felicia Evans, Lancaster, TX (US)

(21) Appl. No.: 17/846,209

(22) Filed: Jun. 22, 2022

(51) Int. Cl. A23L 27/00; A61M 21/00

(52) U.S. Cl. CPC A23L 27/70; A61M 21/00; A61M 2021/0077; A23V 2002/00

(57) ABSTRACT

A dissolving oral implement assembly for facilitating a user with an impaired ability to swallow to enjoy a flavor includes a swab that is comprised of a dissolvable material thereby facilitating the swab to dissolve when the swab is positioned in a user's mouth. The swab is infused with a chemical flavor thereby facilitating the chemical flavor to be released into the user's mouth when the swab is positioned in the user's mouth. In this way the swab can facilitate the user to enjoy a flavor imparted by the chemical flavor. A stem is attached to the swab thereby facilitating the swab to be inserted into the user's mouth.

### CROSS-REFERENCE TO RELATED APPLICATIONS

Not Applicable

### STATEMENT REGARDING FEDERALLY SPONSORED RESEARCH OR DEVELOPMENT Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to oral implement devices and more particularly pertains to a new oral implement device for facilitating a user with an impaired ability to swallow to enjoy a flavor. The device includes a swab comprised of a dissolvable material which is infused with a chemical flavor. The device includes a stem attached to the swab and the stem can be gripped to position the swab in the user's mouth. The device includes a strip, comprised of a dissolvable material, and which is infused with a chemical flavor. The strip can be positioned in the user's mouth thereby dissolving and releasing the chemical flavor into the user's mouth.

### (2) Description of Related Art including information disclosed under 37 CFR 1.97 and 1.98

The prior art relates to oral implement devices including a breath freshener that includes a swab infused with a mouth freshening agent and a stem attached to the swab. The prior art discloses an adult pacifier that includes a concave member, comprised of a resilient material, and a stem. The prior art discloses a flavor pouch which contains a plurality of dissolvable beads that are infused with a chemical flavor. The prior art discloses saliva collection device that includes an absorbent swab coupled to a handle for absorbing and subsequently measuring saliva collected from a user's mouth. The prior art discloses a dissolvable packet filled with an edible composition. The prior art discloses a saliva permeable packet which contains a caffeine bearing substance. The prior art discloses an oral swab which is infused with a proprietary dental care solution.

### BRIEF SUMMARY OF THE INVENTION

An embodiment of the disclosure meets the needs presented above by generally comprising a swab that is comprised of a dissolvable material thereby facilitating the swab to dissolve when the swab is positioned in a user's mouth. The swab is infused with a chemical flavor thereby facilitating the chemical flavor to be released into the user's mouth when the swab is positioned in the user's mouth. In this way the swab can facilitate the user to enjoy a flavor imparted by the chemical flavor. A stem is attached to the swab thereby facilitating the swab to be inserted into the user's mouth.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a dissolving oral implement assembly according to an embodiment of the disclosure.

FIG. 2 is a perspective in use view of swab of an embodiment of the disclosure.

FIG. 3 is a perspective view of strip of an embodiment of the disclosure.

FIG. 4 is a perspective in-use view of strip of an embodiment of the disclosure.

### DETAILED DESCRIPTION OF THE INVENTION

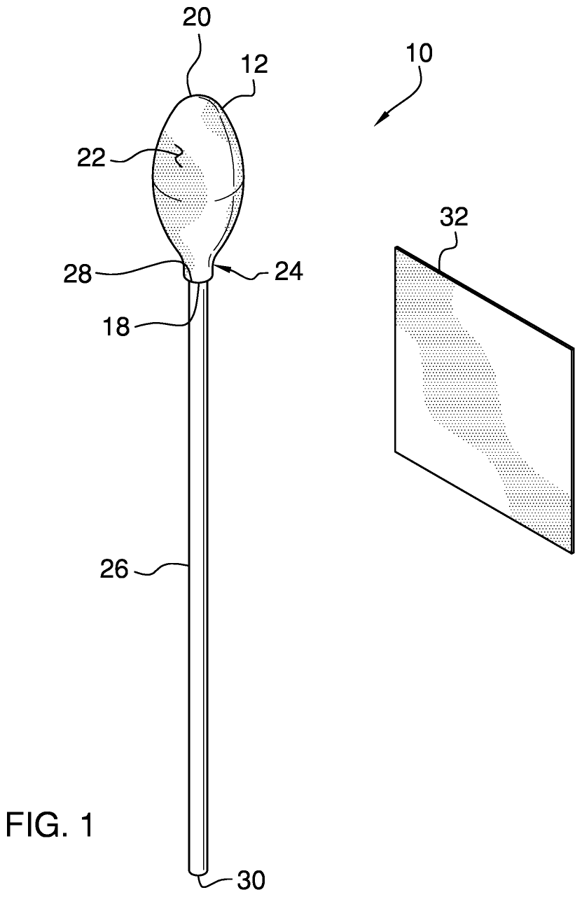

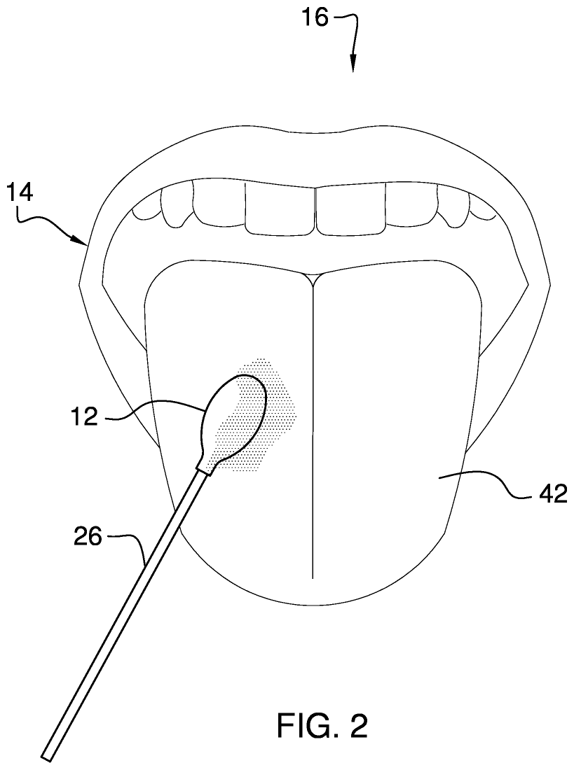

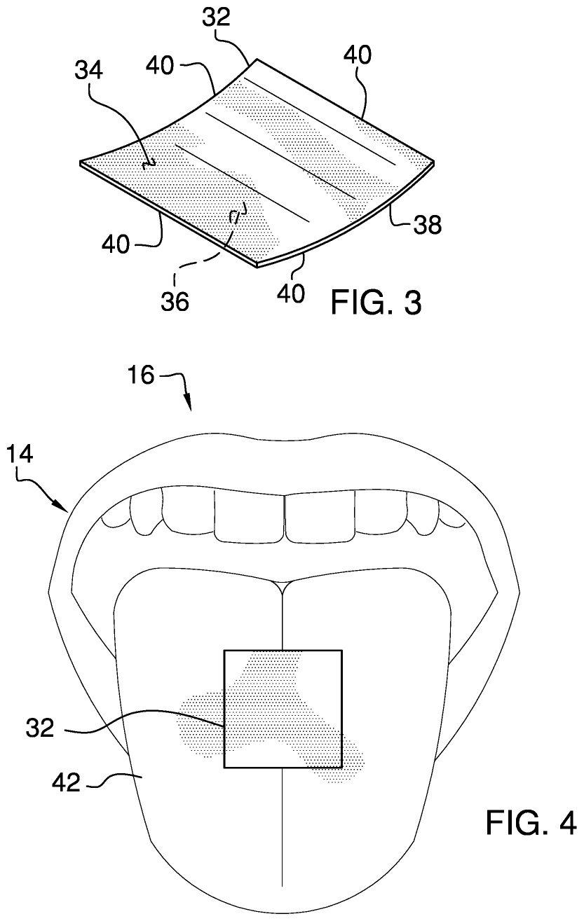

With reference now to the drawings, and in particular to FIGS. 1 through 4 thereof, a new oral implement device embodying the principles and concepts of an embodiment of the disclosure and generally designated by the reference numeral 10 will be described.

As best illustrated in FIGS. 1 through 4, the dissolving oral implement assembly 10 generally comprises a swab 12 that is comprised of a dissolvable material thereby facilitating the swab 12 to dissolve when the swab 12 is positioned in a mouth 14 of a user 16. The swab 12 is infused with a chemical flavor thereby facilitating the chemical flavor to be released into the user's mouth 14 when the swab 12 is positioned in the user's mouth 14. In this way the swab 12 can facilitate the user 16 to enjoy a flavor imparted by the chemical flavor. The user 16 may be a stroke victim or other physically disabled victim that has an impaired ability to swallow. Moreover, the chemical flavor in the swab 12 facilitates the user 16 to enjoy a flavor without having to swallow a beverage or a food item that could potentially threaten the user's 16 health from choking on the food item or aspirating the beverage. The chemical flavor may be a natural fruit flavor, for example, the flavor of coffee, tea or any other flavor that is a favorite flavor of the user 16. The swab 12 may be dipped into a beverage, for example, to soak the swab 12 with the beverage thereby facilitating the swab 12 to release the beverage into the user's mouth 14 thereby facilitating the user 16 to enjoy the flavor of the beverage.

The swab 12 has a first end 18, a second end 20 and an outer surface 22 extending between the first end 18 and the second end 20, and the swab 12 is elongated between the first end 18 and the second end 20. Furthermore, the outer surface 22 is bulbous between the first end 18 and the second end 20 such that the swab 12 has an ovoid shape. The outer surface 22 tapers inwardly adjacent to the first end 18 to define a stem portion 24 of the swab 12 that is not bulbous. Additionally, the first end 18 is flattened and the second end 20 is rounded. The dissolvable material may be a sponge-like material or other deformable, non-rigid material that cannot injure the interior of the user's mouth 14.

A stem 26 is attached to the swab 12 thereby facilitating the swab 12 to be inserted into the user's mouth 14. The stem 26 has a primary end 28 and a secondary end 30, and the stem 26 is elongated between the primary end 28 and the secondary end 30. The primary end 28 is attached to the first end 18 of the swab 12 and the stem 26 may have a length of at least 10.0 cm. The swab 12 may have a length of at least 1.8 cm and a maximum diameter of approximately 0.5 cm.

A strip 32 is comprised of a dissolvable material thereby facilitating the strip 32 to dissolve when the strip 32 is positioned in the user's mouth 14. The strip 32 is infused when a chemical flavor thereby facilitating the chemical flavor in the strip 32 to be released into the user's mouth 14 when the strip 32 is positioned in the user's mouth 14. In this way the strip 32 facilitates the user 16 to enjoy a flavor imparted by the chemical flavor. The strip 32 has an upper surface 34, a lower surface 36 and a perimeter edge 38 extending between the upper surface 34 and the lower surface 36. Furthermore, the perimeter edge 38 has a plurality of intersecting sides 40 such that the strip 32 has a rectangular shape.

In use, the stem 26 is gripped by a caregiver, for example, and the stem 26 is inserted into the user's mouth 14 such that the swab 12 is positioned on the user's tongue 42. In this way the swab 12 will dissolve and release the chemical flavor that is infused in the swab 12 onto the user's tongue 42. The stem 26 is removed from the user's mouth 14 once the swab 12 has dissolved and released the chemical flavor onto the user's tongue 42. The strip 32 can be placed on the user's tongue 42 thereby facilitating the strip 32 to dissolve and release the chemical flavor infused in the strip 32 onto the user's tongue 42.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A dissolving oral implement assembly comprising:
a swab being comprised of a dissolvable material thereby facilitating said swab to dissolve when said swab is positioned in a user's mouth, said swab being infused with a chemical flavor thereby facilitating said chemical flavor to be released into the user's mouth when said swab is positioned in the user's mouth wherein said swab is configured to facilitate the user to enjoy a flavor imparted by, said chemical flavor; and
a stem being attached to said swab thereby facilitating said swab to be inserted into the user's mouth; and
wherein said swab has a first end, a second end and an outer surface extending between said first end and said second end, said swab being elongated between said first end and said second end, said outer surface being bulbous between said first end and said second end such that said swab has an ovoid shape, said outer surface tapering inwardly adjacent to said first end to define a stem portion of said swab, said first end being flattened wherein said first end of said swab defines a lip extending outwardly around said stem, said second end being rounded.

2. (canceled)

3. The dissolving oral implement assembly according to claim 1, wherein said stem has a primary end and a secondary end, said stem being elongated between said primary end and said secondary end, said primary end being attached to said first end of said swab.

4. The dissolving oral implement assembly according to claim 1, further comprising a strip being comprised of a dissolvable material thereby facilitating said strip to dissolve when said strip is positioned in the user's mouth, said strip being infused with said chemical flavor thereby facilitating said chemical flavor in said strip to be released into the user's mouth when said strip is positioned in the user's mouth wherein said configured to facilitate the user to enjoy a flavor imparted by said chemical flavor.

5. The dissolving oral implement assembly according to claim 4, wherein said strip has an upper surface, a lower surface and a perimeter edge extending between said upper surface and said lower surface, said perimeter edge having a plurality of intersecting sides such that said strip has a rectangular shape.

6. A dissolving oral implement assembly comprising:
a swab being comprised of a dissolvable material thereby facilitating said swab to dissolve when said swab is positioned in a user's mouth, said swab being infused with a chemical flavor thereby facilitating said chemical flavor to be released into the user's mouth when said swab is positioned in the user's mouth wherein said swab is configured to facilitate the user to enjoy a flavor imparted by said chemical flavor, said swab having a first end, a second end and an outer surface extending between said first end and said second end, said swab being elongated between said first end and said second end, said outer surface being bulbous between said first end and said second end such that said swab has an ovoid shape, said outer surface tapering inwardly adjacent to said first end to define a stem portion of said swab, said first end being flattened, said second end being rounded;
a stem being attached to said swab thereby facilitating said swab to be inserted into the user's mouth, said stem having a primary end and a secondary end, said stem being elongated between said primary end and said secondary end, said primary end being attached to said first end of said swab, said first end of said swab defining a lip extending outwardly around said stem; and
a strip being comprised of a dissolvable material thereby facilitating said strip to dissolve when said strip is positioned in the user's mouth, said strip being infused with said chemical flavor thereby facilitating said chemical flavor in said strip to be released into the user's mouth when said strip is positioned in the user's mouth wherein said configured to facilitate the user to enjoy said chemical flavor, said strip having an upper surface, a lower surface and a perimeter edge extending between said upper surface and said lower surface, said perimeter edge having a plurality of intersecting sides such that said strip has a rectangular shape.

7. (canceled)
